(19) United States
(12) Patent Application Publication
(10) Pub. No.: US 2011/0003888 A1
(43) Pub. Date: Jan. 6, 2011

(54) HOPS EXTRACTION METHODS AND MIXTURES

(75) Inventor: Eric Kuhrts, Bodega, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bioactives, Inc., Bodega, CA (US)

(21) Appl. No.: 12/522,376

(22) PCT Filed: Jan. 14, 2008
(86) PCT No.: PCT/US08/50996

§ 371 Date: Sep. 22, 2010

Related U.S. Application Data
(60) Provisional application No. 60/884,729, filed on Jan. 12, 2007.

(51) Int. Cl. A61K 31/353; A61K 31/085; A61P 29/00; A61P 31/00; A61P 37/08; A61P 35/00

(52) U.S. Cl. 514/456; 514/720

(57) ABSTRACT

Methods and mixtures for extracting prenylflavonoids from prenylflavonoid-containing hops materials are provided.

### CROSS-REFERENCES TO RELATED APPLICATIONS

This application claims the benefit of U.S. Provisional Patent Application No. 60/884,729, filed Jan. 12, 2007, which is incorporated herein by reference in its entirety for all purposes.

### BACKGROUND OF THE INVENTION

Flavonoids are abundant throughout nature and exert a broad range of biological activities in plants and animals. There are now considered to be over 4,000 flavonoids existent in nature. Some of the biological activities of flavonoids include anti-inflammatory, antiviral, antifungal, antibacterial, estrogenic, anti-oxidant, antiallargenic, anticarcinogenic, and antiproliferative medicinal activities.

Hops (Humulus lupulis L.) has been used for centuries as a bittering agent in the brewing of beer. Hops contains alpha acids such as humulone, co-humuone, ad-humulone, and beta acids such as lupulone and co-lupulone. Hops also contains many flavonoids, such as xanthohumol, isoxanthohumol, desmethylxanthohumol, 8-prenylnaringenin, and 6-prenylnaringenin. Xanthohumol is a yellow-orange substance with a melting point of 172 degrees C. A typical ethanol extract of hops yields about 3 mg./g (3%) of xanthohumol out of a total flavonoid content of 3.46 mg./g. Dried hop contains about 0.2 to 1.0% by weight xanthohumol.

Tannin-free hop products are typically obtained by the extraction of hops with supercritical CO₂, which dissolves bittering and flavoring substances (e.g. α-acids), but virtually no polyphenols.

Xanthohumol and other hop prenylflavonoids have been identified as cancer chemopreventive agents through their interfering action with a variety of cellular mechanisms at low micromolar concentrations such as (1) inhibition of metabolic activation of procarcinogens, (2) induction of carcinogen-detoxifying enzymes, and (3) inhibition of tumor growth by inhibiting inflammatory signals and angiogenesis. Stevens, et al., Phytochemistry 65: 1317-1330 (2004). See also Stevens, et al, Chemistry and Biology of Hops Flavonoids; and Stevens, J. Am. Soc. Brew. Chem. 56(4): 136-145 (1998). Antiproliferative and cytotoxic effects of xanthohumol and five other prenylated hop flavonoids were tested in breast cancer (MCF-7), colon cancer (HT-29), and ovarian cancer (A-2780) cells in vitro. Miranda, et al. Drug Metab. Dispos. 28: 1297-1302 (1999). Xanthohumol inhibited the proliferation of MCF-7 and A-2780 cells in a dose-dependent manner with IC50 values of 13 and 0.52 M, respectively, after two days of treatment. Gerhauser et al. showed that xanthohumol can be an effective anti-inflammatory agent by inhibition of endogenous prostaglandin synthesis through inhibition of cyclooxygenase (constitutive COX-1 and inducible COX-2) enzymes with IC50 values of 17 and 42 μM, respectively. Gerhauser et al., Mol. Cancer. Ther. 1: 959-969 (2002). Xanthohumol, isoxanthohumol, 8-prenylnaringenin, and nine other prenylflavonoids from hops were shown to strongly inhibit the cDNA-expressed human cytochrome P450 enzymes, Cyp1 A1, Cyp1B1, and Cyp1 A2 (Henderson et al., Xenobiotica 30: 235-251 (2000). The effect of 8-prenylnaringenin on angiogenesis was studied by Pepper et al., who demonstrated that 8-prenylnaringenin inhibits angiogenesis in an in vitro model in which endothelial cells can be induced to invade a three-dimensional collagen gel and form capillary-like tubes. Pepper et al., J. Cell Physiol. 199: 98-10 (2004).

Ethanol may be used to extract higher levels of the prenylflavonoids from hops. The typical prenylflavonoid content of an ethanol extract of hops includes xanthohumol (3 mg/g), desmethylxanthohumol (0.34 mg./g), isoxanthohumol (0.052 mg/g), 6-prenylnaringenin (0.061 mg/g), and 8-prenylnaringenin 0.015 (mg/g). Prenylfalvanoids are essentially absent in standard supercritical CO₂ extracts because the prenylflavonoids are virtually insolvent on carbon dioxide.

Due to the many desirable properties of prenylflavonoids, it would be advantageous to have an efficient and economical method of extracting prenylflavonoids from hops. The present invention solves these and other problems in the art.

### BRIEF SUMMARY OF THE INVENTION

In one aspect, the present invention provides a method of extracting a prenylflavonoid from a prenylflavonoid-containing hops material. The method includes the step of contacting the prenylflavonoid-containing hops material with a non-ionic surfactant liquid thereby extracting the prenylflavonoid from the prenylflavonoid-containing hops material.

In another aspect, the present invention provides a prenylflavonoid-containing hops material mixture including a prenylflavonoid-containing hops material in fluid contact with a non-ionic surfactant liquid.

### DETAILED DESCRIPTION OF THE INVENTION

### I. Introduction

It has been discovered that non-ionic surfactant liquids may be used to extract prenylflavonoids from prenylflavonoid-containing hops materials. Thus, the methods described herein provide an unexpected improvement over existing methods of extracting prenylflavonoids from hops materials.

### II. Extraction Method

In one aspect, the present invention provides a method of extracting a prenylflavonoid from a prenylflavonoid-containing hops material. The method includes the step of contacting the prenylflavonoid-containing hops material with a non-ionic surfactant liquid thereby extracting the prenylflavonoid from the prenylflavonoid-containing hops material. The resulting mixture includes a hops biomass material (i.e. the prenylflavonoid-extracted prenylflavonoid-containing hops material) and a prenylflavonoid-containing non-ionic surfactant liquid. In some embodiments, the resulting prenylflavonoid-containing non-ionic surfactant liquid and hops biomass material are filtered in order to separate the prenylflavonoid-containing non-ionic surfactant liquid from the hops biomass material. In other embodiments, water may be added to the prenylflavonoid-containing non-ionic surfactant liquid before, or to facilitate, filtering.

In some embodiments, the non-ionic surfactant liquid is contacted with the prenylflavonoid-containing hops material, optionally heated, and mixed (e.g. mechanically and/or automatically using a shaker, sonicator device, or vortexer) thereby forming a mixture. Where the mixture is heated, the heating temperature is selected to avoid chemical breakdown of the prenylflavonoid and non-ionic surfactant. As used herein, “extracting a prenylflavonoid from a prenylflavonoid-containing hops material” refers to removing some or all prenylflavonoids from a prenylflavonoid-containing hops. The methods of the present invention are carried out by contacting (e.g. mixing, washing, immersing, etc.) the prenylflavonoid-containing hops (typically a solid biomass such as spent hops) with a non-ionic surfactant liquid. The contacting may be performed for any appropriate length of time (e.g. thirty seconds to several hours).

A “prenylflavonoid-containing hops material,” as used herein, refers to a hops or a material derived from hops containing one or more prenylflavonoids. Prenylflavonoids are discussed below in Section V. Hops materials include hop products such as hop cones, hop strobiles, hop glands, and hops which have previously been pre-extracted by supercritical carbon dioxide. In some embodiments, the prenylflavonoid-containing hops material is a xanthohumol-containing hops material and the prenylflavonoid is xanthohumol.

In certain embodiments, the prenylflavonoid-containing hops material is a spent hops. As used herein, “spent hops” refers to hops that have undergone extraction of α-acids and/or β-acids using a prenylflavonoid-insoluble liquid or a prenylflavonoid-insoluble supercritical solvent thereby extracting the α-acids and/or β-acids from the prenylflavonoid-containing hops material.

Useful prenylflavonoid-insoluble liquids and prenylflavonoid-insoluble supercritical solvents are well known in the art. See, for example, U.S. Pat. No. 6,867,332 and U.S. Pat. No. 4,104,409, each of which are incorporated by reference in their entirety for all purposes, particularly the disclosure of useful prenylflavonoid-insoluble liquids and prenylflavonoid-insoluble supercritical solvents. The terms “prenylflavonoid-insoluble liquids” and “prenylflavonoid-insoluble supercritical solvents,” as used herein, mean liquids and supercritical solvents, respectively, that do not solubilize, or solubilize very low amounts (e.g. trace amounts) of prenylflavonoids derived from hops.

For example, the prenylflavonoid-insoluble liquid may be an organic liquid, such as an alkane (e.g. having from 1 to 6 carbon atoms) or petroleum ether. The prenylflavonoid-insoluble liquid may also be liquid carbon dioxide (CO₂). The prenylflavonoid-insoluble supercritical solvent may be selected from CO₂, SF₆, CHF₃, CF₃Cl, CF₂CH₂, CHF₂Cl, C₃F₈, CH₂CH₂, CH₃CH₃ and N₂O. In some embodiments, the prenylflavonoid-insoluble supercritical solvent is a prenylflavonoid-insoluble supercritical CO₂.

A supercritical solvent (sometime referred to in the art as a supercritical fluid) is a substance above its critical temperature and critical pressure. For example, above 31° C. and 73 atm, carbon dioxide behaves as a supercritical fluid and shows properties of both a liquid and a gas. Supercritical carbon dioxide (scCO₂) is an excellent non-polar solvent for many organic compounds. It has been likened to a solvent resembling hexane, though with some hydrogen-bonding acceptor capability and some dipole selectivity. Alkenes, alkanes, aromatics, ketones and alcohols (up to a relative molecular mass of around 400) dissolve in scCO₂. Very polar molecules such as sugars or amino acids and most inorganic salts are insoluble. By adjusting the pressure of the fluid, the solvent properties can be adjusted to be more gas-like or more liquid-like, thereby allowing tuning of the solvent properties.

It is widely known in the art that supercritical CO₂ at high pressures and high temperatures solubilize hops-derived prenylflavonoids. See U.S. Patent Application No. 20040121040, which is herein incorporated by reference in its entirety for all purposes, including the disclosure regarding extraction of prenylflavonoids from hops using supercritical CO₂ at high pressures and high temperatures. Thus, the prenylflavonoid insoluble supercritical CO₂ useful in the present invention is not provided at high pressure and temperature. Thus, in some embodiments, the prenylflavonoid-insoluble supercritical CO₂ is employed at a pressure of less than 500 bar and/or at a temperature of less than 65° C.

### III. Prenylflavonoid-Containing Hops Material Mixtures

In another aspect, the present invention provides a prenylflavonoid-containing hops material mixture including a prenylflavonoid-containing hops material in fluid contact with a non-ionic surfactant liquid. In some embodiments, the prenylflavonoid-containing hops material is immersed, either partially or totally, within the non-ionic surfactant liquid. As described above, upon contacting the prenylflavonoid-containing hops material with a non-ionic surfactant liquid, prenylflavanoid is extracted from the prenylflavonoid-containing hops material. One skilled in the art will recognize that not all of the prenylflavanoid is extracted immediately upon contact. Therefore, the prenylflavonoid-containing hops material mixture is an intermediate in the extraction process of the present invention.

Examples of prenylflavonoids and non-ionic liquids (and non-ionic surfactants) are discussed below, and are equally applicable to both the extraction methods and mixtures of the present invention. The prenylflavonoid-containing hops material may be a xanthohumol-containing hops material. In certain embodiments, the prenylflavonoid-containing hops material is a spent hops.

### IV. Non-Ionic Liquids

In one aspect, the present invention provides a non-ionic surfactant liquid. A non-ionic surfactant liquid is a liquid comprising a non-ionic surfactant in liquid form. In some embodiments, the non-ionic surfactant liquid consists only of a non-ionic surfactant in liquid form. In other embodiments, the non-ionic surfactant liquid is a non-ionic surfactant aqueous solution comprising water and a non-ionic surfactant in liquid form. The non-ionic surfactant may be heated to induce a liquid state. For example, a non-ionic surfactant that is a solid at room temperature may be heated to its melting point thereby forming a non-ionic surfactant liquid. Melting points of non-ionic surfactants are well known in the art.

A “non-ionic surfactant,” as used herein, is a surface active agent that tends to be non-ionized (i.e. uncharged) in neutral solutions (e.g. neutral aqueous solutions). Useful non-ionic surfactants include, for example, non-ionic water soluble mono-, di-, and tri-glycerides; non-ionic water soluble mono- and di-fatty acid esters of polyethyelene glycol; non-ionic water soluble sorbitan fatty acid esters (e.g. sorbitan monooleates such as SPAN 80 and TWEEN 20 (polyoxyethylene 20 sorbitan monooleate)); polyglycolyzed glycerides; non-ionic water soluble triblock copolymers (e.g. poly(ethyleneoxide)/poly-(propyleneoxide)/poly(ethyleneoxide) triblock copolymers such as POLOXAMER 406 (PLURONIC F-127), and derivatives thereof.

Examples of non-ionic water soluble mono-, di-, and tri-glycerides include propylene glycol dicarpylate/dicaprate (e.g. MIGLYOL 840), medium chain mono- and diglycerides (e.g. CAPMUL and IMWITOR72), medium-chain triglycerides (e.g. caprylic and capric triglycerides such as LAVRAFAC, MIGLYOL 810 or 812, CRODAMOL GTCC-PN, and SOFTISON 378), long chain monoglycerides (e.g. glyceryl monooleates such as PECEOL, and glyceryl monolinoleates such as MAISINE), polyoxyl castor oil (e.g. macrogolglycerol ricinoleate, macrogolglycerol hydroxystearate, macrogol cetostearyl ether), and derivatives thereof.

Non-ionic water soluble mono- and di-fatty acid esters of polyethyelene glycol include d-α-tocopheryl polyethyleneglycol 1000 succinate (TPGS), poyethyleneglycol 660 12-hydroxystearate (SOLUTOL HS 15), polyoxyl oleate and stearate (e.g. PEG 400 monostearate and PEG 1750 monostearate), and derivatives thereof.

Polyglycolyzed glycerides include polyoxyethylated oleic glycerides, polyoxyethylated linoleic glycerides, polyoxyethylated caprylic/capric glycerides, and derivatives thereof. Specific examples include LABRAFIL M-1944CS, LABRAFIL M-2125CS, LABRASOL, SOFTIGEN, and GELUCIRE.

In some embodiments, the non-ionic surfactant is a polyoxyl castor oil, or derivative thereof. Effective polyoxyl castor oils may be synthesized by reacting either castor oil or hydrogenated castor oil with varying amounts of ethylene oxide. Macrogolglycerol ricinoleate is a mixture of 83% relatively hydrophobic and 17% relatively hydrophilic components. The major component of the relatively hydrophobic portion is glycerol polyethylene glycol ricinoleate, and the major components of the relatively hydrophilic portion are polyethylene glycols and glycerol ethoxylates. Macrogolglycerol hydroxystearate is a mixture of approximately 75% relatively hydrophobic of which a major portion is glycerol polyethylene glycol 12-oxystearate.

In some embodiments, the non-ionic surfactant aqueous solution is a non-alcoholic solution. A “non-alcoholic” solution, as used herein, is a solution that does not include (or includes only in trace amounts) methanol, ethanol, propanol or butanol. In other embodiments, the formulation does not include (or includes only in trace amounts) ethanol.

In some embodiments, the non-ionic surfactant aqueous solution is a non-aprotic solvated solution. The term “non-aprotic solvated,” as used herein, means that water soluble aprotic solvents are absent or are included only in trace amounts. Water soluble aprotic solvents are water soluble non-surfactant solvents in which the hydrogen atoms are not bonded to an oxygen or nitrogen and therefore cannot donate a hydrogen bond.

In some embodiments, the non-ionic surfactant aqueous solution does not include (or includes only in trace amounts) a polar aprotic solvent. Polar aprotic solvents are aprotic solvents whose molecules exhibit a molecular dipole moment but whose hydrogen atoms are not bonded to an oxygen or nitrogen atom. Examples of polar aprotic solvents include aldehydes, ketones, dimethyl sulfoxide (DMSO), and dimethyl formamide (DMF). In other embodiments, the non-ionic surfactant aqueous solution does not include (or includes only in trace amounts) dimethyl sulfoxide. Thus, in some embodiments, the non-ionic surfactant aqueous solution does not include DMSO or ethanol.

In still other embodiments, the non-ionic surfactant aqueous solution does not include (or includes only in trace amounts) a non-polar aprotic solvent. Non-polar aprotic solvents are aprotic solvents whose molecules exhibit a zero molecular dipole. Examples include hydrocarbons, such as alkanes, alkenes, and alkynes.

### V. Prenylflavonoids

A “prenylflavonoid,” as used herein, refers to a prenylated compound having a phenol attached to a phenyl (e.g. a phenyl substituted with one or more hydroxyl and/or methoxy groups) via a C₃ alkylene group substituted with an oxo (═O) group. The C₃ alkylene may be present in a linear chain arrangement (e.g. a —CH₂═CH₂—C(O)— group found in chalcones) or joined with other atoms to form a substituted or unsubstituted ring (e.g. a dihydro-2H-pyran-4(3H)-one ring found in flavanones). Prenylflavonoids may be derived from natural sources (e.g. hops), or synthesized chemically. Tabat et al., Phytochemistry 46: 683-687 (1997). The term “C₃ alkylene” by itself or as part of another substituent means a divalent radical derived from a three carbon alkyl, as exemplified, but not limited, by —CH₂CH₂CH₂—. The term “alkyl,” by itself or as part of another substituent, means, unless otherwise stated, a straight (i.e. unbranched) or branched chain, or cyclic hydrocarbon radical, or combination thereof, which may be fully saturated, mono- or polyunsaturated and can include di- and multivalent radicals, having the number of carbon atoms designated (i.e. C₃ means three carbons).

As used herein, a “prenylated” compound refers to those compounds with an attached —CH₂—CH═C(CH₃)₂ group (e.g. geranylated compounds), optionally hydroxylated prenyl tautomers (e.g. —CH₂—CH—C(CH₃)═CH₂, or —CH₂—C(OH)—C(CH₃)═CH₂), and optionally hydroxylated circularized prenyl derivatives having the formula:

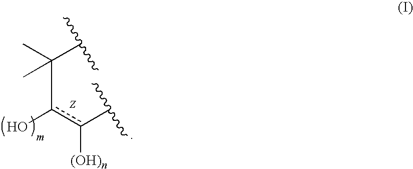

In Formula (I), the dashed bond z represents a double bond or a single bond, and the symbols m and n are independently 0 or 1. The symbol [symbol] represents the point of attachment to the remainder of the prenylated compound.

Thus, prenylflavonoids useful in the present invention include prenylchalcones and/or prenylflavonones. In some embodiments, the prenylflavanoid is a hops prenylflavanoid (i.e. a prenylflavanoid found in hops that is obtained directly from hops or chemically synthesized). In some embodiments, the prenylflavonoid is selected from xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, a8-geranylnaringenin, and metabolites and/or derivatives thereof. In some embodiments, the prenylflavonoid is xanthohumol, a xanthohumol metabolite, or derivatives thereof.

### VI. Assays

In order to effectively extract a prenylflavonoid from a prenylflavonoid-containing hops material, the non-ionic surfactant liquid must be able to solubilize the prenylflavonoid. Thus, subject non-ionic surfactant liquids may be assayed in a preliminary solubilization screening test using any appropriate solubilization test method. Typically, a non-ionic surfactant is contacted with the prenylflavonoid, optionally heated, and mixed (e.g. mechanically and/or automatically using a shaker, sonicator device, or vortexer) to form a solution. Water may be optionally added, for example, where the prenylflavonoid and/or surfactant is in powder form. Where the solution is heated, the heating temperature is selected to avoid chemical breakdown of the prenylflavonoid and non-ionic surfactant.

The resulting solution may be visually inspected for colloidal particles to determine the degree of solubility of the prenylflavonoid. Alternatively, the solution may be filtered and analyzed to determine the degree of solubility. For example, a spectrophotometer may be used to determine the concentration of prenylflavonoid present in the filtered solution. Typically, the test solution is compared to a positive control containing a series of known quantities of pre-filtered prenylflavonoid solutions to obtain a standard concentration versus UV/vis absorbance curve. Alternatively, high performance liquid chromatography may be used to determine the amount of prenylflavonoid in solution.

High throughput solubility assay methods are well known in the art. Typically, these methods involve automated dispensing and mixing of solutions with varying amounts of non-ionic surfactants, prenylflavonoids, and optionally other co-solvents. The resulting solutions may then be analyzed to determine the degree of solubility using any appropriate method as discussed above.

For example, the Millipore MultiScreen Solubility Filter Plate® with modified track-etched polycarbonate, 0.4 μm membrane is a single-use, 96-well product assembly that includes a filter plate and a cover. The device is intended for processing aqueous solubility samples in the 100-300 μL volume range. Thus, when using the aqueous solubility assay, the solution is typically diluted with water. The vacuum filtration design is compatible with standard, microtiter plate vacuum manifolds. The plate is also designed to fit with a standard, 96-well microtiter receiver plate for use in filtrate collection. The MultiScreen Solubility Filter Plate® has been developed and QC tested for consistent filtration flow-time (using standard vacuum), low aqueous extractable compounds, high sample filtrate recovery, and its ability to incubate samples as required to perform solubility assays. The low-binding membrane has been specifically developed for high recovery of dissolved organic compounds in aqueous media.

The aqueous solubility assay allows for the determination of prenylflavonoid solubility by mixing, incubating and filtering a solution in the MultiScreen Solubility filter plate. After the filtrate is transferred into a 96-well collection plate using vacuum filtration, it is analyzed by UV/vis spectroscopy to determine solubility. Additionally, LC/MS or HPLC can be used to determine compound solubility, especially for compounds with low UV/vis absorbance and/or compounds with lower purity. For quantification of aqueous solubility, a standard calibration curve may be determined and analyzed for each compound prior to determining aqueous solubility.

Test solutions may be prepared by adding an aliquot of concentrated drug or compound. The solutions are mixed in a covered 96-well MultiScreen Solubility filter plate for 1.5 hours at room temperature. The solutions are then vacuum filtered into a 96-well, polypropylene, V-bottomed collection plate to remove any insoluble precipitates. Upon complete filtration, 160 μL/well of solution is transferred from the collection plate to a 96-well UV analysis plate and diluted with 40 μL/well of acetonitrile. The UV/vis analysis plate is scanned from 260-500 nm with a UV/vis microplate spectrometer to determine the absorbance profile of the test compound.

Thus, one skilled in the art may screen a wide variety of non-ionic surfactant liquids to determine their ability to solubilize various prenylflavonoid compounds.

After establishing the ability of a non-ionic surfactant to solubilize a prenylflavonoid, the non-ionic surfactant liquid may be assayed for its ability to extract a prenylflavonoid from a prenylflavonoid-containing hops material using an extraction test. The non-ionic surfactant liquid is contacted with a prenylflavonoid-containing hops material. For some non-ionic surfactants, it may be necessary to apply heat in order to achieve a liquid state. After contacting the non-ionic surfactant liquid with a prenylflavonoid-containing hops material, the components are mixed (e.g. mechanically and/or automatically using a shaker, sonicator device, or vortexer) to form a sample mixture. Water may be optionally added to the sample mixture for dilution purposes. The sample mixture may then be filtered to separate the extracted hops biomass material from the non-ionic surfactant liquid sample. The non-ionic surfactant liquid sample is then tested (e.g. via high performance liquid chromatography, NMR, etc.) to determine whether the prenylflavonoid is present.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. All publications, patents, and patent applications cited herein are hereby incorporated by reference in their entirety for all purposes.

### VII. Examples

The examples below are meant to illustrate certain embodiments of the invention, and are intended to limit the scope of the invention.

Lucifer Yellow was purchased from Molecular Probes (Eugene, Oreg.). Hanks buffer and all other chemicals were obtained from Sigma-Aldrich (St. Louis, Mo.).

### Example 1

Water soluble compositions of xanthohumol were formulated containing the non-ionic surfactant macrogolglycerol hydroxystearate. By heating and stirring this polyoxyl castor oil with a powdered xanthohumol extract (containing in excess of 20% xanthohumol), a clear greenish viscous solution was formed containing dissolved xanthohumol (hereinafter referred to as “xanthohumol gel formulation”). The powdered xanthohumol extract consisted of 20% xanthohumol, small amounts of chlorophyll, and uncharacterized residual resins, but did not contain alpha acids, beta acids, or 8-prenylnaringenin. The xanthohumol gel formulation consisted of macrogolglycerol hydroxystearate 40 (100 mL) and powdered xanthohumol extract (10 grams), representing a ratio of surfactant: prenylflavonoid of 10:1.

Water was added to this viscous solution for dilution purposes with solubility being maintained.

An aqueous solution of solubilized xanthohumol was achieved by adding water to this viscous solution (hereinafter referred to as “aqueous xanthohumol formulation”). More specifically, the aqueous xanthohumol formulation was prepared by warming the xanthohumol gel formulation to form a clear aqueous solution of xanthohumol. This aqueous xanthohumol formulation did not have undesirable flavor. The aqueous xanthohumol formulation consisted of water (200 mL), macrogolglycerol hydroxystearate 40 (100 mL), and powdered xanthohumol extract (10 grams), representing a ratio of 20:10:1 for the water:surfactant:prenylflavonoid. The aqueous xanthohumol formulation was analyzed by HPLC and found to contain 0.6%, or 6 mg/mL xanthohumol.

### Example 2

The solubility of the powdered xanthohumol extract in pH 7.4 Hank's Balanced Salt Solution (10 mM HEPES and 15 mM glucose) was compared to the xanthohumol gel formulation. At least 1 mg of powdered xanthohumol extract or 100 mg of xanthohumol gel formulation was combined with 1 mL of buffer to make a≧mg/mL powdered xanthohumol extract mixture and a≧1 mg/mL xanthohumol gel formulation mixture, respectively. The mixtures were shaken for 2 hours using a benchtop vortexer and left to stand overnight at room temperature. After vortexing and standing overnight, the powdered xanthohumol extract mixture was then filtered through a 0.45-μm nylon syringe filter (Whatman, Cat# 6789-0404) that was first saturated with the sample.

The xanthohumol gel formulation mixture was centrifuged at 14,000 rpm for 10 minutes. The filtrate or supernatant was sampled twice, consecutively, and diluted 10, 100, and 10,000-fold in a mixture of 50:50 assay buffer:acetonitrile prior to analysis.

Both mixtures were assayed by LC/MS/MS using electrospray ionization against the standards prepared in a mixture of 50:50 assay buffer:acetonitrile. Standard concentrations ranged from 1.0 μM down to 3.0 nM. Results are presented in Table 1 below.

TABLE 1
Solubility of Xanthohumol in pH 7.4 Phosphate Buffer

| Test Article | Solubility (μM) | | |
|---|---|---|---|
| Identification | Rep 1 | Rep 2 | AVG |
| Powdered Xanthohumol | 0.40 | 0.81 | 0.61 |
| Extract | | | |
| Xanthohumol Gel | 1860 | 1700 | 1780 |
| Formulation | | | |

As shown in Table 1, the powdered xanthohumol extract and xanthohumol gel formulation gel showed average solubility values in pH 7.4 Hank's Balanced Salt Solution of 0.61 μM and 1780 μM, respectively.

### Example 3

The permeability of the xanthohumol gel through a cell-free (blank) microporous 0.4 micron membrane filter was studied in order to determine the non-specific binding and cell-free diffusion Pₐₚₚ of the xanthohumol gel formulation. The xanthohumol gel formulation was assayed at a 2 μM xanthohumol concentration in Hanks buffer (Hanks Balanced Salt Solution (HBSSg) containing 10 mM HEPES and 15 mM glucose) at a pH of 7.4 in duplicate. Donor samples were collected at 120 minutes. Receiver samples were collected at 60 and 120 minutes. The apparent permeability coefficient, Pₐₚₚ, and percent recovery were calculated as follows:

Pₐₚₚ=(dCᵣ/dt)×Vᵣ/(A×C₀)

Percent Recovery=100×((Vᵣ×Cᵣ^{final})+(V_{d}×C_{d}^{final}))/(V_{d}×C₀)
Where:
dCᵣ/dt is the slope of the cumulative concentration in the receiver compartment versus time in μM s⁻¹.
Vᵣ is the volume of the receiver compartment in cm³.
V_{d} is the volume of the donor compartment in cm³.
A is the area of the cell-free insert (1.13 cm² for 12-well Transwell).
Cᵣ^{final} is the cumulative receiver concentration in μM at the end of the incubation period.
C_{d}^{final} is the concentration of the donor in μM at the end of the incubation period.
C₀ is the initial concentration of the dosing solution in μM.

Results of the non-specific binding assessment are presented in Table 2, which shows the permeability (10⁻⁶ cm/s) and recovery of Xanthohumol across the cell-free filter.

TABLE 2

| Xanthohumol Dosing | | | | | |
|---|---|---|---|---|---|
| Solution Concentration (μM) | | Pₐₚₚ (10⁻⁶cm/s) | | | |
| (Average, N = 2) | | A-to-B^{A} | | Recovery (%)^{B} | |
| Rep. 1: | 2.31 | Rep. 1: | 18.6 | Rep. 1: | 95 |
| Rep. 2: | 2.46 | Rep. 2: | 17.1 | Rep. 2: | 99 |
| AVERAGE: | 2.39 | AVERAGE: | 17.9 | AVERAGE: | 97 |

^{A}A low rate of diffusion (<20 × 10⁻⁶ cm/s) through the cell-free membrane may indicate a lack of free diffusion, which may affect the measured permeability.
^{B}Low recoveries caused by non-specific binding, etc. would affect the measured permeability.

### Example 4

To test the permeability of xanthohumol across Caco-2 cell monolayers, Caco-2 cell monolayers were grown to confluence on collagen-coated, microporous, polycarbonate membranes in 12-well Costar Transwell® plates. Details of the plates and their certification are shown below in Table 3. The test article was also the aqueous xanthohumol formulation, and the dosing concentration was 2 μM in the assay buffer (HBSSg) as in the previous example. Cell monolayers were dosed on the apical side (A-to-B) or basolateral side (B-to-A) and incubated at 37° C. with 5% CO₂ in a humidified incubator. Samples were taken from the donor chamber at 120 minutes, and samples from the receiver chamber were collected at 60 and 120 minutes. Each determination was performed in duplicate. Lucifer yellow permeability was also measured for each monolayer after being subjected to the test article to ensure no damage was inflicted to the cell monolayers during the permeability experiment. All samples were assayed for Xanthohumol by LC/MS/MS using electrospray ionization. The apparent permeability (Pₐₚₚ), and percent recovery were calculated as described above. Xanthohumol permeability results are presented in Table 4, which shows the permeability (10⁻⁶ cm/s) and recovery of Xanthohumol across Caco-2 cell monolayers. All monolayers passed the post-experiment integrity control with Lucifer yellow Papp <0.8×10-6 cm/s.

TABLE 3

| Plates | TW12 | |
|---|---|---|
| Seed Date | Jun. 6, 2006 | |
| Passage Number | 63 | |
| Age (Days) | 22 | |
| Parameter | Value | Acceptance Criteria |
| TEER Value (Ω · cm²) | 468 | 450-650 |
| Lucifer Yellow Pₐₚₚ, ×10⁻⁶ cm/s | 0.13 | <0.4 |
| Atenolol Pₐₚₚ, ×10⁻⁶ cm/s | 0.30 | <0.5 |
| Propranolol Pₐₚₚ, ×10⁻⁶ cm/s | 20.65 | 15-25 |
| Digoxin (B-to-A)/(A-to-B) Pₐₚₚ Ratio | 16.57 | >3 |

TABLE 4

| | | Dosing Conc. | Percent | Pₐₚₚ | Efflux | Significant | Absorption |
|---|---|---|---|---|---|---|---|
| Test Article | Direction | (μM) | Recovery^{C} | (10⁻⁶ cm/s) | Ratio | Efflux^{B} | Potential^{A} |
| Xanthohumol | A-to-B | Rep. 1: | Rep. 1: | Rep. 1: | 2.1 | No | Medium |
| | | 2.07 | 30 | 0.94 | | | |
| | | Rep. 2: | Rep. 2: | Rep. 2: | | | |
| | | 2.03 | 28 | 0.74 | | | |
| | | Average | Average: | Average: | | | |
| | | 2.05 | 29 | 0.84 | | | |
| | B-to-A | Rep. 1: | Rep. 1: | Rep. 1: | | | |
| | | 2.25 | 81 | 1.36 | | | |
| | | Rep. 2: | Rep. 2: | Rep. 2: | | | |
| | | 2.21 | 80 | 2.18 | | | |
| | | Average: | Average: | Average: | | | |
| | | 2.23 | 81 | 1.77 | | | |

^{A}Absorption Potential Classification:
Pₐₚₚ(A-to-B) ≧ 1.0 × 10⁻⁶ cm/s High
1.0 × 10⁻⁶ cm/s > Pₐₚₚ(A-to-B) ≧ 0.5 × 10⁻⁶ cm/s Medium
Pₐₚₚ(A-to-B) < 0.5 × 10⁻⁶ cm/s Low
^{B}Efflux considered significant if: Pₐₚₚ(B-to-A) ≧ 1.0 × 10⁻⁶ cm/s and Ratio Pₐₚₚ(B-to-A)/Pₐₚₚ(A-to-B) ≧ 3.0
^{C}Low recoveries caused by non-specific binding, etc. can affect the measured permeability.

### Example 5

The following formulation was prepared as described below: purified xanthohumol 98% (5% by weight), propylene glycol (15% by weight), Flavor (q.s.), povidone (10% by weight), and water (70% by weight).

Propylene glycol was warmed to about 100° F., and the purified xanthohumol (98%) is mixed until a clear yellowish solution is obtained. The warm mixture was slowly added to the water while mixing. Finally, the povidone and flavor was added.

### Example 6

The following formulation was prepared as described below: 8-prenylnaringenin 98% (10% by weight), macrogolglycerol hydroxystearate 40 (90% by weight).

The macrogolglycerol hydroxystearate 40 was warmed until clear. The 8-prenylnaringenin was slowly mixed or vortexed into solution until invisible. The resulting solution was clear. This clear solution is optionally added to water and flavored to create a pleasant tasting beverage, or encapsulated into a soft gel capsule.

### Example 7

Macrogolglycerol hydroxystearate 40 powder was heated to approximately 100° F. while stirring or vortexing until clear. Prenylflavonoid were extracted from spent hops powder (whole hops flowers extracted with supercritical CO₂ and then dried) by contacting the heated macrogolglycerol hydroxystearate 40 liquid with a powdered spent hops and mixing thoroughly by vortexing until the powder was approximately uniformly dispersed in solution. The solution was then diluted with water and filtered to separate the extracted spent hops biomass thereby producing a clear liquid extract. The clear liquid extract was subjected to high performance liquid chromatography. Peaks corresponding to prenylflavonoid elution times were detected thereby confirming the presence of extracted prenylflavonoids in the clear liquid extract.

What is claimed is:

1. A method of extracting a prenylflavonoid from a prenylflavonoid-containing hops material, said method comprising the step of: (a) contacting said prenylflavonoid-containing hops material with a non-ionic surfactant liquid thereby extracting said prenylflavonoid from said prenylflavonoid-containing hops material.

2. The method of claim 1, wherein said prenylflavonoid is selected from the group consisting of xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, and 8-geranylnaringenin.

3. The method of claim 1, wherein said prenylflavonoid-containing hops material is a xanthohumol-containing hops material and said prenylflavonoid is xanthohumol.

4. The method of claim 1, wherein said prenylflavonoid-containing hops material is a spent hops.

5. The method of claim 1, wherein prior to step (a), said method comprises the step of: (a1) contacting said prenylflavonoid-containing hops material with a prenylflavonoid-insoluble liquid or a prenylflavonoid-insoluble supercritical solvent thereby extracting α-acid and β-acid from said prenylflavonoid-containing hops material.

6. The method of claim 3, wherein said contacting in step (a1) is with said prenylflavonoid-insoluble supercritical solvent.

7. The method of claim 4, wherein said prenylflavonoid-insoluble supercritical solvent is a prenylflavonoid-insoluble supercritical carbon dioxide solvent.

8. The method of claim 7, wherein said prenylflavonoid-insoluble supercritical carbon dioxide solvent is at a pressure of less than 500 bar and at a temperature of less than 65° C.

9. The method of claim 1, wherein said non-ionic surfactant liquid is a non-ionic water soluble mono-, di-, or tri-glyceride; non-ionic water soluble mono- or di-fatty acid ester of polyethyelene glycol; non-ionic water soluble sorbitan fatty acid ester; polyglycolyzed glyceride; non-ionic water soluble triblock copolymers, or derivative thereof.

10. The method of claim 1, wherein said non-ionic surfactant liquid is a non-ionic water soluble mono-, di-, or tri-glyceride.

11. The method of claim 1, wherein said non-ionic surfactant liquid is polyoxyl castor oil.

12. The method of claim 1, wherein said non-ionic surfactant liquid is macrogolglycerol ricinoleate or macrogolglycerol hydroxystearate.

13. The method of claim 1, wherein said non-ionic surfactant liquid is macrogolglycerol hydroxystearate.

14. The method of claim 1, wherein said non-ionic surfactant liquid comprises a non-ionic surfactant and water.

15. A prenylflavonoid-containing hops material mixture comprising a prenylflavonoid-containing hops material in fluid contact with a non-ionic surfactant liquid.

16. The mixture of claim 15, wherein said prenylflavonoid-containing hops material comprises a prenylflavonoid selected from the group consisting of xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, and 8-geranylnaringenin.

17. The mixture of claim 15, wherein said prenylflavonoid-containing hops material is a xanthohumol-containing hops material.

18. The mixture of claim 15, wherein said prenylflavonoid-containing hops material is a spent hops.

19. The mixture of claim 15, wherein said non-ionic surfactant liquid is a non-ionic water soluble mono-, di-, or tri-glyceride; non-ionic water soluble mono- or di-fatty acid ester of polyethyelene glycol; non-ionic water soluble sorbitan fatty acid ester; polyglycolyzed glyceride; non-ionic water soluble triblock copolymers, or derivative thereof.

20. The mixture of claim 15, wherein said non-ionic surfactant liquid is a non-ionic water soluble mono-, di-, or tri-glyceride.

21. The mixture of claim 15, wherein said non-ionic surfactant liquid is polyoxyl castor oil.

22. The mixture of claim 15, wherein said non-ionic surfactant liquid is macrogolglycerol ricinoleate or macrogolglycerol hydroxystearate.

23. The mixture of claim 15, wherein said non-ionic surfactant liquid is macrogolglycerol hydroxystearate.

24. The mixture of claim 15, wherein said non-ionic surfactant liquid comprises a non-ionic surfactant and water.
